SEQUENCE LISTING (84 sequences)

SEQ ID NO: 1 - DNA, 9 bp, Homo sapiens
aagcctaag

SEQ ID NO: 2 - DNA, 10 bp, Homo sapiens
taagcctaag

SEQ ID NO: 3 - DNA, 11 bp, Homo sapiens
gtaagcctaag

SEQ ID NO: 4 - DNA, 12 bp, Homo sapiens
agtaagcctaag

SEQ ID NO: 5 - DNA, 13 bp, Homo sapiens
gagtaagcctaag

SEQ ID NO: 6 - DNA, 14 bp, Homo sapiens
cgagtaagcctaag

SEQ ID NO: 7 - DNA, 15 bp, Homo sapiens
acgagtaagcctaag

SEQ ID NO: 8 - DNA, 16 bp, Homo sapiens
aacgagtaagcctaag

SEQ ID NO: 9 - DNA, 17 bp, Homo sapiens
gaacgagtaagcctaag

SEQ ID NO: 10 - DNA, 18 bp, Homo sapiens
ggaacgagtaagcctaag

SEQ ID NO: 11 - DNA, 9 bp, Homo sapiens
aagcctaac

SEQ ID NO: 12 - DNA, 10 bp, Homo sapiens
taagcctaac

SEQ ID NO: 13 - DNA, 11 bp, Homo sapiens
gtaagcctaac

SEQ ID NO: 14 - DNA, 12 bp, Homo sapiens
agtaagcctaac

SEQ ID NO: 15 - DNA, 13 bp, Homo sapiens
gagtaagcctaac

SEQ ID NO: 16 - DNA, 14 bp, Homo sapiens
cgagtaagcctaac

SEQ ID NO: 17 - DNA, 15 bp, Homo sapiens
acgagtaagcctaac

SEQ ID NO: 18 - DNA, 16 bp, Homo sapiens
aacgagtaagcctaac

SEQ ID NO: 19 - DNA, 17 bp, Homo sapiens
gaacgagtaagcctaac

SEQ ID NO: 20 - DNA, 18 bp, Homo sapiens
ggaacgagtaagcctaac

SEQ ID NO: 21 - DNA, 9 bp, Homo sapiens
gctgagcgg

SEQ ID NO: 22 - DNA, 10 bp, Homo sapiens
ggctgagcgg

SEQ ID NO: 23 - DNA, 11 bp, Homo sapiens
aggctgagcgg

SEQ ID NO: 24 - DNA, 12 bp, Homo sapiens
aaggctgagcgg

SEQ ID NO: 25 - DNA, 13 bp, Homo sapiens
caaggctgagcgg

SEQ ID NO: 26 - DNA, 14 bp, Homo sapiens
ccaaggctgagcgg

SEQ ID NO: 27 - DNA, 15 bp, Homo sapiens
tccaaggctgagcgg

SEQ ID NO: 28 - DNA, 16 bp, Homo sapiens
atccaaggctgagcgg

SEQ ID NO: 29 - DNA, 17 bp, Homo sapiens
catccaaggctgagcgg

SEQ ID NO: 30 - DNA, 18 bp, Homo sapiens
acatccaaggctgagcgg

SEQ ID NO: 31 - DNA, 9 bp, Homo sapiens
gctgagcga

SEQ ID NO: 32 - DNA, 10 bp, Homo sapiens
ggctgagcga

SEQ ID NO: 33 - DNA, 11 bp, Homo sapiens
aggctgagcga

SEQ ID NO: 34 - DNA, 12 bp, Homo sapiens
aaggctgagcga

SEQ ID NO: 35 - DNA, 13 bp, Homo sapiens
caaggctgagcga

SEQ ID NO: 36 - DNA, 14 bp, Homo sapiens
ccaaggctgagcga

SEQ ID NO: 37 - DNA, 15 bp, Homo sapiens
tccaaggctgagcga

SEQ ID NO: 38 - DNA, 16 bp, Homo sapiens
atccaaggctgagcga

SEQ ID NO: 39 - DNA, 17 bp, Homo sapiens
catccaaggctgagcga

SEQ ID NO: 40 - DNA, 18 bp, Homo sapiens
acatccaaggctgagcga

SEQ ID NO: 41 - DNA, 9 bp, Homo sapiens
gcgttgaat

SEQ ID NO: 42 - DNA, 10 bp, Homo sapiens
agcgttgaat

SEQ ID NO: 43 - DNA, 11 bp, Homo sapiens
tagcgttgaat

SEQ ID NO: 44 - DNA, 12 bp, Homo sapiens
ctagcgttgaat

SEQ ID NO: 45 - DNA, 13 bp, Homo sapiens
tctagcgttgaat

SEQ ID NO: 46 - DNA, 14 bp, Homo sapiens
ttctagcgttgaat

SEQ ID NO: 47 - DNA, 15 bp, Homo sapiens
cttctagcgttgaat

SEQ ID NO: 48 - DNA, 16 bp, Homo sapiens
tcttctagcgttgaat

SEQ ID NO: 49 - DNA, 17 bp, Homo sapiens
ttcttctagcgttgaat

SEQ ID NO: 50 - DNA, 18 bp, Homo sapiens
gttcttctagcgttgaat

SEQ ID NO: 51 - DNA, 19 bp, Homo sapiens
tgttcttctagcgttgaat

SEQ ID NO: 52 - DNA, 20 bp, Homo sapiens
gtgttcttctagcgttgaat

SEQ ID NO: 53 - DNA, 9 bp, Homo sapiens
gcgttgaag

SEQ ID NO: 54 - DNA, 10 bp, Homo sapiens
agcgttgaag

SEQ ID NO: 55 - DNA, 11 bp, Homo sapiens
tagcgttgaag

SEQ ID NO: 56 - DNA, 12 bp, Homo sapiens
ctagcgttgaag

SEQ ID NO: 57 - DNA, 13 bp, Homo sapiens
tctagcgttgaag

SEQ ID NO: 58 - DNA, 14 bp, Homo sapiens
ttctagcgttgaag

SEQ ID NO: 59 - DNA, 15 bp, Homo sapiens
cttctagcgttgaag

SEQ ID NO: 60 - DNA, 16 bp, Homo sapiens
tcttctagcgttgaag

SEQ ID NO: 61 - DNA, 17 bp, Homo sapiens
ttcttctagcgttgaag

SEQ ID NO: 62 - DNA, 18 bp, Homo sapiens
gttcttctagcgttgaag

SEQ ID NO: 63 - DNA, 19 bp, Homo sapiens
tgttcttctagcgttgaag

SEQ ID NO: 64 - DNA, 20 bp, Homo sapiens
gtgttcttctagcgttgaag

SEQ ID NO: 65 - DNA, 9 bp, Homo sapiens
cacacagtg

SEQ ID NO: 66 - DNA, 10 bp, Homo sapiens
gcacacagtg

SEQ ID NO: 67 - DNA, 11 bp, Homo sapiens
tgcacacagtg

SEQ ID NO: 68 - DNA, 12 bp, Homo sapiens
gtgcacacagtg

SEQ ID NO: 69 - DNA, 13 bp, Homo sapiens
agtgcacacagtg

SEQ ID NO: 70 - DNA, 14 bp, Homo sapiens
aagtgcacacagtg

SEQ ID NO: 71 - DNA, 15 bp, Homo sapiens
gaagtgcacacagtg

SEQ ID NO: 72 - DNA, 16 bp, Homo sapiens
tgaagtgcacacagtg

SEQ ID NO: 73 - DNA, 17 bp, Homo sapiens
atgaagtgcacacagtg

SEQ ID NO: 74 - DNA, 18 bp, Homo sapiens
gatgaagtgcacacagtg

SEQ ID NO: 75 - DNA, 9 bp, Homo sapiens
cacacagta

SEQ ID NO: 76 - DNA, 10 bp, Homo sapiens
gcacacagta

SEQ ID NO: 77 - DNA, 11 bp, Homo sapiens
tgcacacagta

SEQ ID NO: 78 - DNA, 12 bp, Homo sapiens
gtgcacacagta

SEQ ID NO: 79 - DNA, 13 bp, Homo sapiens
agtgcacacagta

SEQ ID NO: 80 - DNA, 14 bp, Homo sapiens
aagtgcacacagta

SEQ ID NO: 81 - DNA, 15 bp, Homo sapiens
gaagtgcacacagta

SEQ ID NO: 82 - DNA, 16 bp, Homo sapiens
tgaagtgcacacagta

SEQ ID NO: 83 - DNA, 17 bp, Homo sapiens
atgaagtgcacacagta

SEQ ID NO: 84 - DNA, 18 bp, Homo sapiens
gatgaagtgcacacagta

(19) United States
(12) Patent Application Publication
(10) Pub. No.: US 2008/0280843 A1
(43) Pub. Date: Nov. 13, 2008

(54) Methods and kits for linking polymorphic sequences to expanded repeat mutations

(76) Inventors: Paul van Bilsen, Maastricht (NL); William F. Kaemmerer, Edina, MN (US); Eric Burright, Eagan, MN (US)

Correspondence Address: FOX ROTHSCHILD LLP;PRINCETON PIKE CORPORATE CENTER, 2000 Market Street, Tenth Floor, Philadelphia, PA, 19103, US

(21) Appl. No.: 11/439,858

(22) Filed: May 24, 2006

(51) Int. Cl. A61K 31/7105; C12Q 1/68

(52) U.S. Cl. 514/44; 435/6

(57) ABSTRACT

Methods and kits are provided for determining which single nucleotide polymorphism (“SNP”) variant of an allele of a heterozygous patient is on the same mRNA transcript as a disease-causing mutation that is at a remote region of the gene's mRNA comprising a) an allele-specific reverse transcription reaction using an allele-specific primer, and b) analysis of the resulting cDNA product from the reverse transcription reaction at the region of the mutation to determine the presence or absence of the mutation on this allele-specific cDNA product.

### FIELD OF THE INVENTION

The present invention relates generally to compositions and methods for diagnosing diseases which have an allele-specific therapy and a disease-causing mutation that is sufficiently distant from the molecular site of the therapy to require a diagnostic linking method.

### BACKGROUND OF THE INVENTION

Expansions of CAG trinucleotide repeats (CAG repeats) in coding regions of human genes cause numerous disorders by generating proteins with elongated polyglutamine (polyQ) stretches. This group of disorders includes by way of example Dystophia myotonica, Spinocerebellar ataxia type 1, Spinocerebellar ataxia type 2, Spinocerebellar ataxia type 3, Spinocerebellar ataxia type 6, Spinocerebellar ataxia type 7, Spinocerebellar ataxia type 8, Spinocerebellar ataxia type 17, Huntington disease-like 2, Spinal and bulbar muscular atrophy, Huntington disease, Dentatorubral-pallidoluysian atrophy, Oculopharyngeal dystrophy, Congenital central hypoventilation syndrome, Infantile spasms, Synpolydactyl), Cleidocranial dysplasia, Holoprosencephaly, Hand-foot-genital syndrome, Type II blephorophimosis, ptosis, and epicanthus inversus syndrome. (Wanker E. E. (2000) Biol. Chem., 381:937-942; Gusella J. F. and MacDonald, M. E. (2000) Nature Rev. Neurosci., 1:109-115; and Usdin K. and Grabczyk, E. (2000) Cell. Mol. Life Sci., 57:914-931).

For purposes of illustration only Huntington's disease (HD) will be discussed herein. HD is a chronic neurodegenerative disorder which is inherited as an autosomal dominant trait and is characterized by chorea, dementia and personality disorder. Martin, J. B. and Gusella, J. F. (1986) N. Engl. J. Med. 315:1267-1276. The gene responsible for HD contains an expanded and unstable CAG trinucleotide repeat. Huntington's Disease Collaborative Research Group (1993) Cell 72:971-983.

The HD gene (IT15 gene), which encodes huntingtin, a 350 kDa protein of unknown function, is located on the human chromosome 4 and consists of 67 exons. The disease-causing mutation is a CAG repeat expansion located within exon 1 of the HD gene (HD exon1). The CAG repeat is translated into a polyQ stretch. The disease manifests itself when the polyQ stretch exceeds the critical length of 37 glutamines (pathological threshold), whereas 8-35 glutamine residues in huntingtin are tolerated by neuronal cells. Experimental evidence has been presented that huntingtin fragments with polyQ tracts in the pathological range (more than 37 glutamines), but not in the normal range (20-32 glutamines), form high molecular weight protein aggregates with a fibrillar morphology in vitro and in cell culture model systems (Scherzinger et al. (1999) Proc. Natl. Acad. Sci. USA, 96:4604-4609; and Waelter et al., (2001) Mol. Biol. Cell, 12:1393-1407). In addition, inclusions with aggregated N-terminally truncated huntingtin protein were detected in HD transgenic mice carrying a CAG repeat expansion of 115-156 units and in HD patient brains (Davies et al., (1997) Cell, 90:537-548; and DiFiglia et al., (1997) Science, 277:1990-1993), suggesting that the process of aggregate formation may be important for the progression of HD. The mechanisms, however, by which the elongated polyQ sequences in huntingtin cause dysfunction and neurodegeneration are not yet understood (Scherzinger et al., (1999); Tobin A. J. and Signer, E. R. (2000) Trends Cell Biol., 10:531-536; and Perutz M. F. (1999) Glutamine repeats and neurodegenerative diseases: molecular aspects. Trends Biochem. Sci., 24:58-63).

Unaffected individuals have repeat numbers of up to 30, while individuals at a high risk of developing HD carry more than 37 CAG repeats. Individuals with 30-37 repeats have a high risk of passing on repeats in the affected size range to their offspring (Andrew et al., (1997) Hum. Mol. Genet., 6:2005-2010; Duyao et al., (1993) Nature Genet., 4:387-392; and Snell et al., (1993) Nature Genet., 4:393-397).

It is known that patients are able to survive and live healthy lives with only one functioning copy of the IT15 gene. Thus, selective inactivation of the allele with a disease-causing mutation should diminish or even eliminate the disease while improving the possibilities of survival in heterozygous patients.

The combination of emotional, cognitive and motor symptoms in HD contributes to an unusually high cost of care. People with Huntington's Disease require care from health professionals of many stripes, including general practitioners, neurologists, social workers, home health aides, psychologists, physical therapists, and speech/language pathologists.

Currently, there are a few diagnostic approaches for nucleic acid sequence identification. U.S. Patent Application Publication No. 20040048301 describes allele-specific primer extension in the presence of labeled nucleotides for sequence identification, but does not include allele-specific primer extension for enrichment of one allele over the other for further analysis of the allele of interest as part of the kit. WO Patent Application No. 2003100101 describes isolation of one sequence in a mixture by hybridization markers and single-strand specific nucleases for use in single-molecule analysis. U.S. Patent Application Publication No. 20030039964 describes a method for isolation of one sequence in a mixture by hybridization to a fixed probe, but does not disclose the use of reverse transcription. U.S. Pat. No. 6,013,431 describes a method for analysis of bases adjacent to a hybridized, immobilized oligo, but does not disclose enrichment of one allele over the other. WO Patent Application No. 9820166 describes a method for specific selection of one allele over the other, followed by mass spectroscopic analysis of the selected molecule, but does not disclose the use of reverse transcription. None of these references disclose methods and diagnostic kits for linking polymorphic sequences to expanded repeat mutations for improved allele-specific diagnosis and allele-specific gene therapies.

### SUMMARY OF THE INVENTION

Applicants have invented methods and kits for determining which variant allele of a single nucleotide polymorphism (“SNP”) located at a distance from a disease-causing mutation co-segregates with the disease-causing mutation. In other words, the invention will allow for the determination of which SNP allele is located on the same mRNA transcript as the transcribed disease-causing mutation.

In one aspect, the invention provides a method for determining which single nucleotide polymorphism variant of an allele from a gene isolated from a heterozygous patient is on the same mRNA transcript as a disease-causing mutation at a remote region of the gene's mRNA comprising: a) an allele-specific reverse transcription reaction using an allele-specific primer which recognizes one single nucleotide polymorphism variant, and b) analysis of an allele-specific cDNA product from the allele-specific reverse transcription reaction at the remote region of the gene to determine the presence or absence of the mutation on the allele-specific cDNA product. In one embodiment, the 3′ end of the allele-specific primer is positioned at the single nucleotide polymorphism nucleotide position.

In another aspect, the invention provides a method of treating a patient comprising determining which SNP variant is on the same mRNA transcript as a disease-causing mutation according to the method recited above, and applying an allele-specific therapy to the SNP variant. The allele-specific therapy of the present invention includes by way of example allele-specific RNA interference using siRNA or shRNA.

In yet another aspect, the invention provides a kit for determining which single nucleotide polymorphism variant of an allele of a heterozygous patient is on the same mRNA transcript as a disease-causing mutation located at a remote region of the gene's mRNA comprising a) an allele-specific primer which recognizes one single nucleotide polymorphism variant, and b) a set of instructions. In one embodiment, the 3′ end of the allele-specific primer is positioned at the single nucleotide polymorphism nucleotide position.

### DETAILED DESCRIPTION OF THE INVENTION

The present invention relates to methods and kits for performing allele-specific reverse transcription from an SNP site and analysis of a cDNA at a region of gene mutation. The methods, systems and reagents of the present invention are applicable to any disease which contains an SNP variant of an allele in a heterozygous subject that is on the same mRNA transcript as a disease-causing mutation that is at a remote region of the gene's mRNA.

To aid in the understanding of the invention, the following non-limiting definitions are provided:

The term “gene” refers to a DNA sequence that comprises control and coding sequences necessary for the production of a polypeptide or its precursor. The polypeptide can be encoded by a full length coding sequence (either genomic DNA or cDNA) or by any portion of the coding sequence so long as the desired activity is retained. In some aspects, the term “gene” also refers to an mRNA sequence or a portion thereof that directly codes for a polypeptide or its precursor.

The term “transfection” refers to the uptake of foreign DNA by a cell. A cell has been “transfected” when exogenous (i.e., foreign) DNA has been introduced inside the cell membrane. Transfection can be either transient (i.e., the introduced DNA remains extrachromosomal and is diluted out during cell division) or stable (i.e., the introduced DNA integrates into the cell genome or is maintained as a stable episomal element).

“Cotransfection” refers to the simultaneous or sequential transfection of two or more vectors into a given cell.

The term “promoter element” or “promoter” refers to a DNA regulatory region capable of being bound by an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences.

The term “in operable combination”, “in operable order” or “operably linked” refers to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The term “vector” refers to a nucleic acid assembly capable of transferring gene sequences to target cells (e.g., viral vectors, non-viral vectors, particulate carriers, and liposomes). The term “expression vector” refers to a nucleic acid assembly containing a promoter which is capable of directing the expression of a sequence or gene of interest in a cell. Vectors typically contain nucleic acid sequences encoding selectable markers for selection of cells that have been transfected by the vector. Generally, “vector construct,” “expression vector,” and “gene transfer vector,” refer to any nucleic acid construct capable of directing the expression of a gene of interest and which can transfer gene sequences to target cells. Thus, the term includes cloning and expression vehicles, as well as viral vectors.

The term “antibody” refers to a whole antibody, both polyclonal and monoclonal, or a fragment thereof, for example a F(ab)₂, Fab, FV, VH or VK fragment, a single chain antibody, a multimeric monospecific antibody or fragment thereof, or a bi- or multi-specific antibody or fragment thereof. The term also includes humanized and chimeric antibodies.

The term “treating” or “treatment” of a disease refers to executing a protocol, which may include administering one or more drugs to a patient (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols which have only a marginal effect on the patient.

The term “patient” refers to a biological system to which a treatment can be administered. A biological system can include, for example, an individual cell, a set of cells (e.g., a cell culture), an organ, a tissue, or a multi-cellular organism. A patient can refer to a human patient or a non-human patient.

The terms “remote region” or “remote location” indicate a distance of at least 100 base pairs from the SNP site toward the transcription initiation.

The term “practitioner” refers to a person who uses methods, kits and compositions of the current invention on the patient. The term includes, without limitations, doctors, nurses, scientists, and other medical or scientific personnel.

The terms “siRNA molecule,” “shRNA molecule,” “RNA molecule,” “DNA molecule,” “cDNA molecule” and “nucleic acid molecule” are each intended to cover a single molecule, a plurality of molecules of a single species, and a plurality of molecules of different species.

The term “siRNA” refers to a double-stranded RNA molecule wherein each strand is between about 15 and about 30 bases of ribonucleic acid in length, and the two strands have a region of complementarity such that the two strands hybridize or “base pair” together through the annealing of complementary bases (Adenosine to Uracil, and Guanine to Cytosine). For some siRNA molecules, the two strands hybridize together in a manner such that there is an overhang of non-annealed bases at the 5′ or 3′ ends of the strand. For other siRNA molecules, the two strands hybridize together such that each base of one strand is paired with a base of the other strand. For some siRNA molecules, the two strands may not be 100% complementary, but may have some bases that do not hybridize due to a mismatch. For some siRNA molecules, the RNA bases may be chemically modified, or additional chemical moieties may be conjugated to one or more ends of one or more of the strands.

The term “shRNA” refers to a “short, hairpin” RNA molecule comprised of a single strand of RNA bases that self-hybridizes in a hairpin structure. The RNA molecule is comprised of a stem region of RNA bases that hybridize together to form a double-stranded region, and a loop region of RNA bases that form the bend of the hairpin. The term “shRNA” also refers to a DNA molecule from which a short, hairpin RNA molecule may be transcribed in vitro or in vivo.

The methods of the present invention utilize routine techniques in the field of molecular biology. Basic texts disclosing general molecular biology methods include Sambrook et al., Molecular Cloning, A Laboratory Manual (3d ed. 2001) and Ausubel et al., Current Protocols in Molecular Biology (1994).

The present invention relates generally to compositions and methods for diagnosing diseases which have an allele-specific therapy and a disease-causing mutation that is sufficiently distant from the molecular site of the therapy. Table 1 depicts certain diseases applicable to the present invention. Table 1 was derived from information previously published (DiProspero (2005)). Table 1 describes in part examples of triplet repeat expansion diseases and the mutant gene associated with each disease.

TABLE 1
Triplet Repeat Expansion Disorders

| | | | | |
|---|---|---|---|---|
| Disease | Symptoms | Gene | Locus | Protein |

Non-coding repeats

| | | | | |
|---|---|---|---|---|
| Dystrophia | Weakness, | DMPK | 19q13 | Dystrophia |
| myotonica 1 | Myotonia | | | myotonica |
| | | | | Protein kinase |
| Spinocerebellar | Ataxia | Antisense | 13q21 | Undetermined |
| ataxia 8 | | to KLHL1 | | |
| Huntington disease- | Chorea, dementia | JPH3 | 16q24.3 | Junctophilin 3 |
| like2 | | | | |

Polyglutamine disorders

| | | | | |
|---|---|---|---|---|
| Spinal and bulbar | Weakness | AR | Xq13-q21 | Androgen |
| muscular atrophy | | | | receptor |
| Huntington disease | Chorea, dementia | IT15 | 4P16.3 | Huntingtin |
| Dentatorubral- | Ataxia, myoclonic | DRPLA | 12p13.31 | Atrophin 1 |
| pallidoluysian | epilepsy, | | | |
| atrophy | dementia | | | |
| Spinocerebellar | Ataxia | SCA1 | 6p23 | Ataxin 1 |
| ataxia 1 | | | | |
| Spinocerebellar | Ataxia | SCA2 | 12q24.1 | Ataxin 2 |
| ataxia 2 | | | | |
| Spinocerebellar | Ataxia | SCA3/MJD | 14q32.1 | Ataxin 3 |
| ataxia 3 (Machado- | | | | |
| Joseph disease) | | | | |
| Spinocerebellar | Ataxia | CACNA1A | 19p13 | α_{1A}-voltage- |
| ataxia 6 | | | | dependent |
| | | | | calcium |
| | | | | channel |
| | | | | subunit |
| Spinocerebellar | Ataxia | SCA7 | 3p12-p13 | Ataxin 7 |
| ataxia 7 | | | | |
| Spinocerebellar | Ataxia | TBP | 6q27 | TATA box |
| ataxia 17 | | | | binding |
| | | | | protein |

Polyalanine disorders*

| | | | | |
|---|---|---|---|---|
| Oculopharyngeal | Weakenss | PABPN1 | 14q11.2-q13 | Poly(A)- |
| dystrophy | | | | binding |
| | | | | protein 2 |
| Congential central | Respiratory | PHOX2B | 4p12 | Paired-like |
| hypoventilation | difficulties | | | homeobox 2B |
| syndrome | | | | |
| Infantile spasms | Mental | ARX | Xp22.13 | Aristaless- |
| | retardation, | | | related |
| | epilepsy | | | homeobox, X- |
| | | | | linked |
| Synpolydactyly | Limb malformation | HOXD13 | 2q31-q32 | Homeobox D13 |

*Polyalanine expansions have also been reported among mutations in other genes, including RUNX2 (runt-related transcription factor2) in cleidocranial dysplasia, ZIC2 (Zic family member 2) in holoprosencephaly HOXA13 (homeobox A13) in hand-foot-genital syndrome, and FOXL2 (forkhead box L2) in type II blepharophimosis, ptosis, and epicanthus inversus syndrome. Small aspartic acid repeat expansions have been reported among other mutations in the COMP (cartilage oligomeric mat4rix protein) gene in patients with multiple epiphyseal dysplasia.

The present invention is not limited to the diseases described above. There may be situations where a disease is caused by many different mutations in a single gene (thus designing many different gene-targeting therapies may not be practical from a commercial perspective). However, if one or two expressed SNPs are present in the disease-associated gene, then the SNPs may actually serve as the molecular target for the therapy (and thus determination of linkage of the SNP to the disease-causing mutation would be essential).

For purposes of illustration, only HD will be discussed herein as an example of a triplet repeat expansion disease and example of the applicability of the present invention in providing methods and kits for determining allele-specific reverse transcription from an SNP site and analysis of a cDNA at a region of mutation.

The coding region of the IT15 gene is about 13,000 bases long. The HD disease-causing mutation is the expansion of the CAG repeat region. The CAG repeat region starts at about nucleotide position 15. If the CAG triplets repeat for about 25 or 30 times, the patient is not at risk of the disease. If however, more than 37 CAG repeats occur in a row on the nucleotide sequence then the patient is going to get Huntington's disease.

About ten thousand bases downstream from the CAG repeat sequence, there is a natural variation (Single Nucleotide Polymorphism, or SNP) of the IT15 gene in the human population, where for many people it might be an “A residue” and for many others it is a “C residue”. That is just a normal variation, as it does not cause any disease. The information about the SNP can be used to determine that a child of a Huntington's disease patient has inherited an allele with the “A residue” from one parent and an allele with the “C residue” from the other parent.

The practitioner also knows that one of the patient's parents has HD and would like to know if the patient will also get HD. The practitioner can actually determine whether the patient is going to get HD or not, by looking at both of the patient's IT15 alleles, and determining how many CAG repeats the gene contains. If one of the CAG repeats is longer than 37, then the patient will get HD. Further, the practitioner can determine whether the patient is heterozygous (i.e., one allele has a normal number of repeats, e.g., 20, while the other allele has expanded repeats, e.g., 37). Analyzing the IT15 gene downstream of the CAG repeats, the practitioner may find that the patient received a “C residue” from one parent and an “A residue” from the other parent. Thus, the crucial issue for the allele-specific diagnosis is which SNP is on the same mRNA transcript as the expanded number of repeats in the patient's IT15 gene. Isolating the genetic information from the patient's parents may not help because it is possible that one or both parents are also heterozygous (e.g., each parent has two SNP variants of the gene (i.e., an A residue and a C residue variants). This disclosure provides a method of determining which SNP allele of the gene co-segregates with the disease-causing mutation.

One aspect of the present invention provides a diagnostic test, allowing the practitioner to determine which allele, classified by the nucleotide at the SNP position, co-segregates with the disease-causing mutation. In one embodiment, the test comprises a method for determining which single nucleotide polymorphism variant of an allele from a gene isolated from a heterozygous patient is on the same mRNA transcript as a disease-causing mutation at a remote region of the gene's mRNA comprising: a) an allele-specific reverse transcription reaction using an allele-specific primer which recognizes one single nucleotide polymorphism variant, wherein the 3′ end of the allele-specific primer is positioned at the single nucleotide polymorphism nucleotide position, and b) analysis of an allele-specific cDNA product from the allele-specific reverse transcription reaction at the remote region of the gene to determine the presence or absence of the mutation on the allele-specific cDNA product.

In a layman's terms, the practitioner takes RNA from the patient and applies a reverse transcription primer that recognizes just the “A allele.” The “A allele” specific primer will have at its 3′ position a complement to the SNP variant of interest. In case of the A-variant, the “A allele” specific primer will have the T at the 3′ end, and so when this “A allele” specific primer anneals to the mRNA, it will base-pair with the 3′ end and allow the reverse transcriptase to proceed to synthesize the cDNA from the “A allele.” Conversely, the “A allele” primer will not base-pair at the 3′ end of the primer with the “C allele” (since T is not complementary to C). Thus, the reverse transcription polymerase will not be able to produce cDNA from the C allele. On the other hand, in the “A” portion of a reaction, the practitioner will obtain a pool of the cDNAs that corresponds to the “A allele.” The reaction can be repeated in a separate tube with a “C allele” specific primer and no “A allele” primer. A person of ordinary skill in the art will understand that the “C allele” specific primer will have a G on its 3′ end. Essentially the practitioner will perform at least one allele-specific reverse transcription reaction, but preferably two allele-specific reverse transcriptions reactions (each with its own allele-specific primer), on the mRNA from the patient. As a result, the practitioner will have two sub-populations of cDNA, wherein each subpopulation is allele-specific, and the practitioner knows which pool corresponds to which variant. Thus, the practitioner will be able to use any number of possible methods, the simplest being PCR to analyze the upstream portion of the cDNA containing the CAG repeat region and quantify the number of the repeats from the cDNA products that came specifically from the “C reaction” or specifically from the “A reaction.”

The embodiment of the invention described above employs the notion that a mismatch on the 3′ position of the allele-specific primer will not allow reverse transcriptase to produce cDNA from the allele with a mismatched SNP variant. A person of ordinary skill in the art will undoubtedly recognize that the 3′ end of the allele-specific primer does not have to be positioned at the single nucleotide polymorphism nucleotide position. For example, a skilled artisan may design primers and conditions of the reverse transcription reaction in such a way that the allele-specific primer will not bind altogether and thus lead to the same end result: absence of cDNA the allele with a mismatched SNP variant.

The accurate determination of the number of CAG repeats is required for the DNA-based predictive testing of at-risk individuals. To date, CAG repeat length determination is based on polymerase chain reaction (PCR) amplification of genomic DNA using primers flanking the CAG repeat region in the IT15 gene, and subsequent electrophoretic separation of the products in denaturing polyacrylamide gels (Williams et al., (1999) Comparative semi-automated analysis of (CAG) repeats in the HD gene: use of internal standards. Mol. Cell. Probes, 13:283-289).

Numerous methods and commercial kits for the synthesis of first strand cDNA molecules are well known in the art. Examples include the Superscript™ Double Strand cDNA Synthesis Kit (Invitrogen, Carlsbad, Calif.), the Array 50™, Array 350™ and Array 900™ Detection Kits (Genisphere, Hatfield, Pa.), and the CyScribe™ Post-Labelling Kit (Amersham, Piscataway, N.J.). RNA molecules (e.g., mRNA, hnRNA, rRNA, tRNA, miRNA, snoRNA, non-coding RNAs) from a source of interest are used as templates in a reverse transcription reaction. The RNA may be obtained from a mammalian or more preferably human tissue or cell source. The methods of the present invention are particularly suited for amplification of RNA from small numbers of cells, including single cells, which can be purified from complex cellular samples using, e.g., micromanipulation, fluorescence-activated cell sorting (FACS) and laser microdissection techniques (see Player et al., Expert Rev. Mol. Diagn. 4:831 (2004)).

Any reverse transcriptase can be used in the initial reverse transcription reaction, including thermostable, RNAse H⁺ and RNase H⁻ reverse transcriptases. Preferably, an RNase H⁻ reverse trancriptase is used.

Primers for first strand cDNA synthesis can be obtained commercially or synthesized and purified using techniques well known in the art.

PCR amplifications of the CAG repeat region have primarily been performed by incorporating [α-³²P]dNTPs, or using ³²P or fluorescently end-labeled primers. Sizing of fluorescently end-labeled amplification products was performed in various Applied Biosystems DNA sequencers (Andrew et al., (1993) Nature Genet., 4:398-403; Choudhry et al., (2001) Hum. Mol. Genet., 10:2437-2446; Ishii et al., (2001) J. Clin. Endocrinol. Metab., 86:5372-5378; Le et al., (1997) Mol. Pathol., 50:261-265; Mangiarini et al., (1997) Nature Genet., 15:197-200; Pelotti et al., (2001) Am. J. Forensic Med. Pathol., 22:55-57; Wallerand et al., (2001) Fertil. Steril., 76:769-774; Warner et al., (1993) Mol. Cell Probes, 7:235-239; and Warner et al., (1996) J. Med. Genet., 33:1022-1026).

High-resolution method can be used for the exact length determination of CAG repeats in HD genes as well as in genes affected in related CAG repeat disorders (Elisabeth Möncke-Buchner et al., Nucleic Acids Res. 2002 Aug. 15; 30(16)).

A wide variety of kits may be prepared according to present invention. For example, a kit may include a single stranded promoter template comprising at least one RNA polymerase recognition sequence; and instructional materials for synthesizing cDNA molecules using said promoter template. While the instructional materials typically comprise written or printed materials, they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. Such media may include addresses to internet sites that provide such instructional materials.

The kits of the present invention may further include one or more of the following components or reagents: a reverse transcriptase (preferably with DNA-dependent DNA polymerase activity); an RNase inhibitor; an enzyme for attaching a 3′ oligodeoxynucleotide tail onto DNA molecules (e.g., terminal deoxynucleotidyl transferase); an enzyme for degrading RNA in RNA/DNA duplexes (e.g., RNase H); and one or more RNA polymerases (e.g., T7, T3 or SP6 RNA polymerase). Additionally, the kits may include buffers, primers (e.g., oligodT primers, random primers), nucleotides, labeled nucleotides, an RNase inhibitor, polyA polymerase, RNase-free water, containers, vials, reaction tubes, and the like compatible with the synthesis of sRNA molecules according to the methods of the present invention. The components and reagents may be provided in containers with suitable storage media.

A person of ordinary skill in the art will appreciate that such allele-specific diagnosis empowers a practitioner to devise and implement an allele-specific treatment which generally comprises inactivation of the mutated copy of the gene. It is known that patients are able to survive and live healthy lives with only one functioning copy of the HD gene. It is known that the expression of the mutant gene is causing the trouble for the HD patient. Applicants' therapeutic model provides for selectively shutting off mutant gene expression without affecting expression of the normal gene, and is applicable to any disease which contains an SNP variant of an allele in a heterozygous subject that is on the same mRNA transcript as a disease-causing mutation that is at a remote region of the gene's mRNA.

Accordingly, another aspect of the present invention provides a method of treating a patient susceptible to Huntington's disease comprising: a) determining which single nucleotide polymorphism variant is on the same mRNA transcript as a disease-causing mutation according to an allele-specific reverse transcription reaction using an allele-specific primer which recognizes one single nucleotide polymorphism variant, wherein further the 3′ end of the primer is positioned at the single nucleotide polymorphism nucleotide position, and b) analysis of the resulting cDNA product from the reverse transcription reaction at the region of the mutation to determine the presence or absence of the mutation on this allele-specific cDNA product, and c) applying an allele-specific therapy to the SNP variant. It should be noted that the allele-specific therapy could itself operate at a different SNP site than the SNP site used to make the determination about which allele contains the mutation, so long as the SNP site of the therapy target and the SNP site used to identify the mutation-containing allele are already determined, before the therapy is administered to the patient, to be linked; that is, on the same mRNA transcript.

In some embodiments of the present invention the allele-specific therapy comprises allele-specific RNA interference using siRNA or shRNA. In this embodiment of the invention, the allele-specific therapy destroys the “A allele” of the patient. In this embodiment the siRNA targets the “A allele” upon introduction into the subject's brain by any method known to those of skill in the art (See for example, U.S. application Ser. No. 11/253,393, U.S. application Ser. No. 10/852,997, U.S. application Ser. No. 10/721,693, U.S. application Ser. No. 11/157,608, and PCT Patent Application No. US05/022156, which are incorporated herein in their entirety). When the siRNA is delivered into a cell it is used by proteins in the cell (known as the RISC complex) to find and destroy the mRNA from the Huntington's gene that has the “A allele.” Thus, the messenger RNA is destroyed before it can be used to make protein. Conversely, the allele that came from the healthy parent does not get destroyed and so its messenger RNA still survives to be used to make functional biologically active protein.

The design and use of small interfering RNA complementary to mRNA targets that produce particular proteins is a recent tool employed by molecular biologists to prevent translation of specific mRNAs. Various groups have been recently studying the effectiveness of siRNAs as biologically active agents for suppressing the expression of specific proteins involved in neurological disorders. Caplen et al. Human Molecular Genetics, 11(2): 175-184 (2002) assessed a variety of different double stranded RNAs for their ability to inhibit cell expression of mRNA transcripts of the human androgen receptor gene containing different CAG repeats. Their work found gene-specific inhibition occurred with double stranded RNAs containing CAG repeats only when flanking sequences to the CAG repeats were present in the double stranded RNAs. They were also able to show that constructed double stranded RNAs were able to rescue caspase-3 activation induced by expression of a protein with an expanded polyglutamine region. Xia, Mao, et al., Nature Biotechnology, 20: 1006-1010 (2002) demonstrated the inhibition of polyglutamine (CAG) expression in engineered neural PC12 clonal cell lines that express a fused polyglutamine-fluorescent protein using constructed recombinant adenovirus expressing siRNAs targeting the mRNA encoding green fluorescent protein.

One aspect of the present invention provides an siRNA molecule corresponding to at least a portion of a gene containing an SNP variant of an allele in a heterozygous subject that is on the same mRNA transcript as a disease-causing mutation located at a remote region of the gene's mRNA, wherein such siRNA nucleic acid sequence is capable of inhibiting expression of the mRNA transcript containing the disease-causing mutation in a cell. siRNAs are typically short (19-29 nucleotides), double-stranded RNA molecules that cause sequence-specific degradation of complementary target mRNA known as RNA interference (RNAi). Bass, Nature 411:428 (2001).

Accordingly, in some embodiments, the siRNA molecules comprise a double-stranded structure comprising a sense strand and an antisense strand, wherein the antisense strand comprises a nucleotide sequence that is complementary to at least a portion of a desired nucleic acid sequence and the sense strand comprises a nucleotide sequence that is complementary to at least a portion of the nucleotide sequence of said antisense region, and wherein the sense strand and the antisense strand each comprise about 19-29 nucleotides.

Any desired nucleic acid sequence can be targeted by the siRNA molecules of the present invention. Nucleic acid sequences encoding desired gene targets are publicly available from Genbank.

The siRNA molecules targeted to desired sequence can be designed based on criteria well known in the art (e.g., Elbashir et al., EMBO J. 20:6877 (2001)). For example, the target segment of the target mRNA preferably should begin with AA (most preferred), TA, GA, or CA; the GC ratio of the siRNA molecule preferably should be 45-55%; the siRNA molecule preferably should not contain three of the same nucleotides in a row; the siRNA molecule preferably should not contain seven mixed G/Cs in a row; the siRNA molecule preferably should comprise two nucleotide overhangs (preferably TT) at each 3′ terminus; the target segment preferably should be in the ORF region of the target mRNA and preferably should be at least 75 bp after the initiation ATG and at least 75 bp before the stop codon; and the target segment preferably should not contain more than 16-17 contiguous base pairs of homology to other coding sequences.

Based on some or all of these criteria, siRNA molecules targeted to desired sequences can be designed by one of skill in the art using the aforementioned criteria or other known criteria (e.g., Gilmore et al., J. Drug Targeting 12:315 (2004); Reynolds et al., Nature Biotechnol. 22:326 (2004); Ui-Tei et al., Nucleic Acids Res. 32:936 (2004)). Such criteria are available in various web-based program formats useful for designing and optimizing siRNA molecules (e.g., siDESIGN Center at Dharmacon; BLOCK-iT RNAi Designer at Invitrogen; siRNA Selector at Wistar Institute; siRNA Selection Program at Whitehead Institute; siRNA Design at Integrated DNA Technologies; siRNA Target Finder at Ambion; and siRNA Target Finder at Genscript).

siRNA molecules targeted to desired sequences can be produced in vitro by annealing two complementary single-stranded RNA molecules together (one of which matches at least a portion of a desired nucleic acid sequence) (e.g., U.S. Pat. No. 6,506,559) or through the use of a short hairpin RNA (shRNA) molecule which folds back on itself to produce the requisite double-stranded portion (Yu et al., Proc. Natl. Acad. Sci. USA 99:6047 (2002)). Such single-stranded RNA molecules can be chemically synthesized (e.g., Elbashir et al., Nature 411:494 (2001)) or produced by in vitro transcription using DNA templates (e.g., Yu et al., Proc. Natl. Acad. Sci. USA 99:6047 (2002)). When chemically synthesized, chemical modifications can be introduced into the siRNA molecules to improve biological stability. Such modifications include phosphorothioate linkages, fluorine-derivatized nucleotides, deoxynucleotide overhangs, 2′-O-methylation, 2′-O-allylation, and locked nucleic acid (LNA) substitutions (Dorset and Tuschl, Nat. Rev. Drug Discov. 3:318 (2004); Gilmore et al., J. Drug Targeting 12:315 (2004)).

siRNA molecules targeted to desired target sequences can be introduced into cells to inhibit expression. Alternatively, DNA molecules from which shRNA molecules targeted to desired target sequences can be introduced into cells to inhibit expression. Accordingly, another aspect of the present invention provides for inhibiting expression of an mRNA sequence containing an SNP allele and a disease-causing mutation in a cell comprising introducing into a cell at least one siRNA molecule or shRNA molecule that corresponds to at least a portion of the mRNA nucleic acid sequence. Any cell can be targeted. For example, the siRNA or shRNA molecules are introduced into a heart cell or brain cell. In some embodiments, the brain cell is from a subject at risk for HD, i.e., the offspring of a HD patient.

The siRNA molecules produced herein can be introduced into cells in vitro or ex vivo using techniques well-known in the art, including electroporation, calcium phosphate co-precipitation, microinjection, lipofection, polyfection, and conjugation to cell penetrating peptides (CPPs). The siRNA molecules can also be introduced into cells in vivo by direct delivery into specific organs such as the liver, brain, eye, lung and heart, or systemic delivery into the blood stream or nasal passage using naked siRNA molecules or siRNA molecules encapsulated in biodegradable polymer microspheres (Gilmore et al., J. Drug Targeting 12:315 (2004)).

Alternatively, siRNA molecules targeted to specific mRNA sequences can be introduced into cells in vivo by endogenous production from an expression vector(s) encoding the sense and antisense siRNA sequences. Accordingly, another aspect of the present invention provides an expression vector comprising at least one DNA sequence encoding a siRNA molecule corresponding to at least a portion of a specific mRNA nucleic acid sequence capable of inhibiting expression of a specific mRNA in a cell operably linked to a genetic control element capable of directing expression of the siRNA molecule in a cell. Expression vectors can be transfected into cells using any of the methods described above.

Genetic control elements include a transcriptional promoter, and may also include transcription enhancers to elevate the level of mRNA expression, a sequence that encodes a suitable ribosome binding site, and sequences that terminate transcription. Suitable eukaryotic promoters include constitutive RNA polymerase II promoters (e.g., cytomegalovirus (CMV) promoter, the SV40 early promoter region, the promoter contained in the 3′ long terminal repeat of Rous sarcoma virus (RSV), the herpes thymidine kinase (TK) promoter, and the chicken beta-actin promoter), cardiac-tissue-specific RNA polymerase II promoters (e.g., the ventricular myosin light chain 2 (MLC-2v) promoter, and the sodium-calcium exchanger gene H1 promoter (NCX1H1)), and RNA polymerase III promoters (e.g., U6, H1, 7SK and 7SL).

In some embodiments, the sense and antisense strands of siRNA molecules are encoded by different expression vectors (i.e., cotransfected) (e.g., Yu et al., Proc. Natl. Acad. Sci. USA 99:6047 (2002). In other embodiments, the sense and antisense strands of siRNA molecules are encoded by the same expression vector. The sense and antisense strands can be expressed separately from a single expression vector, using either convergent or divergent transcription (e.g., Wang et al., Proc. Natl. Acad. Sci. USA 100:5103 (2003); Tran et al., BMC Biotechnol. 3:21 (2003)). Alternatively, the sense and antisense strands can be expressed together from a single expression vector in the form of a single hairpin RNA molecule, either as a short hairpin RNA (shRNA) molecule (e.g., Arts et al., Genome Res. 13:2325 (2003)) or a long hairpin RNA molecule (e.g., Paddison et al., Proc. Natl. Acad. Sci. USA 99:1443 (2002)).

Although numerous expression vectors can be used to express siRNA molecules in cells (Dorsett and Tuschl, Nat. Rev. Drug Discov. 3:318 (2004)), viral expression vectors are preferred, particularly those that efficiently transduce heart cells (e.g., alphaviral, lentiviral, retroviral, adenoviral, adeno-associated viral (AAV)) (Williams and Koch, Annu. Rev. Physiol. 66:49 (2004); del Monte and Hajjar, J. Physiol. 546.1:49 (2003). Both adenoviral and AAV vectors have been shown to be effective at delivering transgenes (including transgenes directed to diseases) into heart, including failing cardiomycoytes (e.g., Iwanaga et al., J. Clin. Invest. 113:727 (2004); Seth et al., Proc. Natl. Acad. Sci. USA 101:16683 (2004); Champion et al., Circulation 108:2790 (2003); Li et al., Gene Ther. 10:1807 (2003); Vassalli et al., Int. J. Cardiol. 90:229 (2003); del Monte et al., Circulation 105:904 (2002); Hoshijima et al., Nat. Med. 8:864 (2002); Eizema et al., Circulation 101:2193 (2000); Miyamoto et al., Proc. Natl. Acad. Sci. USA 97:793 (2000); He et al., Circulation 100:974 (1999). Recent reports have demonstrated the use of AAV vectors for sustained gene expression in mouse and hamster myocardium and arteries for over one year (Li et al., Gene Ther. 10:1807 (2003); Vassalli et al., Int. J. Cardiol. 90:229 (2003)). In particular, expression vectors based on AAV serotype 6 have been shown to efficiently transduce both skeletal and cardiac muscle (e.g., Blankinship et al., Mol. Ther. 10:671 (2004)). The present invention also provides for the use of coxsackie viral vectors for delivery of desired siRNA sequences.

Following introduction of the desired siRNA molecules into cells, changes in desired gene product levels can be measured if desired. Desired gene products include, for example, desired mRNA and desired polypeptide, and both can be measured using methods well-known to those skilled in the art. For example, desired mRNA can be directly detected and quantified using, e.g., Northern hybridization, in situ hybridization, dot and slot blots, or oligonucleotide arrays, or can be amplified before detection and quantitation using, e.g., polymerase chain reaction (PCR), reverse-transcription-PCR (RT-PCR), PCR-enzyme-linked immunosorbent assay (PCR-ELISA), or ligase chain reaction (LCR).

Desired polypeptide (or fragments thereof) can be detected and quantified using various well-known immunological assays, such as, e.g., enzyme-linked immunosorbent assay (ELISA), radioimmunoassay (RIA), immunoprecipitation, immunofluorescence, and Western blotting. Anti-desired antibodies (preferably anti-human desired) for use in immunological assays are commercially available from, e.g., EMD Biosciences (San Diego, Calif.), Upstate (Charlottesville, Va.), Abcam (Cambridge, Mass.), Affinity Bioreagents (Golden, Colo.) and Novus Biologicals (Littleton, Colo.), or may be produced by methods well-known to those skilled in the art.

Specific embodiments according to the methods of the present invention will now be described in the following examples. The examples are illustrative only, and are not intended to limit the remainder of the disclosure in any way.

### EXAMPLES

### Example 1

RNA-isolation and Reverse transcription reaction. Applicants analyzed the CAG-repeat sequences in the Huntington's disease gene using the following allele-specific reverse transcription reaction. Table 2 defines the sequences for various allele-specific reverse transcription primers for use in determining which allele of a heterozygous patient's Huntington's disease gene contains the disease-causing allele, in accordance with the subject invention. The subject SNP sites in the Huntington's disease gene (IT15) are designated using the identification number provided by the National Center for Biotechnology Information (NCBI) database, accessible at: http://www.ncbi.nlm.nih.gov/entrez/query.fcgi?db=snp.

TABLE 2
SNP NCBI: rs1936032

| Allele C | | | Allele G | | |
|---|---|---|---|---|---|
| SEQ. ID. | Name | Sequence | SEQ. ID. | Name | Sequence |
| 1 | 1936032C9 | AAGCCTAAG | 11 | 1936032G9 | AAGCCTAAC |
| 2 | 1936032C10 | TAAGCCTAAG | 12 | 1936032G10 | TAAGCCTAAC |
| 3 | 1936032C11 | GTAAGCCTAAG | 13 | 1936032G11 | GTAAGCCTAAC |
| 4 | 1936032C12 | AGTAAGCCTAAG | 14 | 1936032G12 | AGTAAGCCTAAC |
| 5 | 1936032C13 | GAGTAAGCCTAAG | 15 | 1936032G13 | GAGTAAGCCTAAC |
| 6 | 1936032C14 | CGAGTAAGCCTAAG | 16 | 1936032G14 | CGAGTAAGCCTAAC |
| 7 | 1936032C15 | ACGAGTAAGCCTAAG | 17 | 1936032G15 | ACGAGTAAGCCTAAC |
| 8 | 1936032C16 | AACGAGTAAGCCTAAG | 18 | 1936032G16 | AACGAGTAAGCCTAAC |
| 9 | 1936032C17 | GAACGAGTAAGCCTAAG | 19 | 1936032G17 | GAACGAGTAAGCCTAAC |
| 10 | 1936032C18 | GGAACGAGTAAGCCTAAG | 20 | 1936032G18 | GGAACGAGTAAGCCTAAC |

SNP NCBI: rs2530588

| Allele C | | | Allele T | | |
|---|---|---|---|---|---|
| SEQ. ID. | Name | Sequence | SEQ. ID. | Name | Sequence |
| 21 | 2530588C9 | GCTGAGCGG | 31 | 2530588T9 | GCTGAGCGA |
| 22 | 2530588C10 | GGCTGAGCGG | 32 | 2530588T10 | GGCTGAGCGA |
| 23 | 2530588C11 | AGGCTGAGCGG | 33 | 2530588T11 | AGGCTGAGCGA |
| 24 | 2530588C12 | AAGGCTGAGCGG | 34 | 2530588T12 | AAGGCTGAGCGA |
| 25 | 2530588C13 | CAAGGCTGAGCGG | 35 | 2530588T13 | CAAGGCTGAGCGA |
| 26 | 2530588C14 | CCAAGGCTGAGCGG | 36 | 2530588T14 | CCAAGGCTGAGCGA |
| 27 | 2530588C15 | TCCAAGGCTGAGCGG | 37 | 2530588T15 | TCCAAGGCTGAGCGA |
| 28 | 2530588C16 | ATCCAAGGCTGAGCGG | 38 | 2530588T16 | ATCCAAGGCTGAGCGA |
| 29 | 2530588C17 | CATCCAAGGCTGAGCGG | 39 | 2530588T17 | CATCCAAGGCTGAGCGA |
| 30 | 2530588C18 | ACATCCAAGGCTGAGCGG | 40 | 2530588T18 | ACATCCAAGGCTGAGCGA |

SNP NCBI: rs363125

| Allele A | | | Allele C | | |
|---|---|---|---|---|---|
| SEQ. ID. | Name | Sequence | SEQ. ID. | Name | Sequence |
| 41 | 363125A9 | GCGTTGAAT | 53 | 363125C9 | GCGTTGAAG |
| 42 | 363125A10 | AGCGTTGAAT | 54 | 363125C10 | AGCGTTGAAG |
| 43 | 363125A11 | TAGCGTTGAAT | 55 | 363125C11 | TAGCGTTGAAG |
| 44 | 363125A12 | CTAGCGTTGAAT | 56 | 363125C12 | CTAGCGTTGAAG |
| 45 | 363125A13 | TCTAGCGTTGAAT | 57 | 363125C13 | TCTAGCGTTGAAG |
| 46 | 363125A14 | TTCTAGCGTTGAAT | 58 | 363125C14 | TTCTAGCGTTGAAG |
| 47 | 363125A15 | CTTCTAGCGTTGAAT | 59 | 363125C15 | CTTCTAGCGTTGAAG |
| 48 | 363125A16 | TCTTCTAGCGTTGAAT | 60 | 363125C16 | TCTTCTAGCGTTGAAG |
| 49 | 363125A17 | TTCTTCTAGCGTTGAAT | 61 | 363125C17 | TTCTTCTAGCGTTGAAG |
| 50 | 363125A18 | GTTCTTCTAGCGTTGAAT | 62 | 363125C18 | GTTCTTCTAGCGTTGAAG |
| 51 | 363125A19 | TGTTCTTCTAGCGTTGAAT | 63 | 363125C19 | TGTTCTTCTAGCGTTGAAG |
| 52 | 363125A20 | GTGTTCTTCTAGCGTTGAAT | 64 | 363125C20 | GTGTTCTTCTAGCGTTGAAG |

SNP NCBI: rs362331

| Allele C | | | Allele T | | |
|---|---|---|---|---|---|
| SEQ. ID. | Name | Sequence | SEQ. ID. | Name | Sequence |
| 65 | 362331C9 | CACACAGTG | 75 | 362331T9 | CACACAGTA |
| 66 | 362331C10 | GCACACAGTG | 76 | 362331T10 | GCACACAGTA |
| 67 | 362331C11 | TGCACACAGTG | 77 | 362331T11 | TGCACACAGTA |
| 68 | 362331C12 | GTGCACACAGTG | 78 | 362331T12 | GTGCACACAGTA |
| 69 | 362331C13 | AGTGCACACAGTG | 79 | 362331T13 | AGTGCACACAGTA |
| 70 | 362331C14 | AAGTGCACACAGTG | 80 | 362331T14 | AAGTGCACACAGTA |
| 71 | 362331C15 | GAAGTGCACACAGTG | 81 | 362331T15 | GAAGTGCACACAGTA |
| 72 | 362331C16 | TGAAGTGCACACAGTG | 82 | 362331T16 | TGAAGTGCACACAGTA |
| 73 | 362331C17 | ATGAAGTGCACACAGTG | 83 | 362331T17 | ATGAAGTGCACACAGTA |
| 74 | 362331C18 | GATGAAGTGCACACAGTG | 84 | 362331T18 | GATGAAGTGCACACAGTA |

### Materials and Methods

Splitting the cell line. HD 4022 Fibroblast cells were grown in an 80 cm² culture bottle in a minimum essential medium (MEM+Earle's salts+2 mM L-Glutamine, Invitrogen 31095-029), with 20% FBS (Fetal Bovine Serum, Invitrogen 10109-155), 1% antibiotics (Penicillin-Streptomycin-Neomycin (PSN), Invitrogen 15640-055), 1% fungizone (Fungizone, Invitrogen 15290-026), 2% non-essential amino acids (Non-essential amino acids, Invitrogen 11130-036), 2% amino acids (Amino acids, Invitrogen 11140-035), and 2% vitamins (Vitamins, Invitrogen 11120-037). The medium was removed and the cells were washed with 10 ml 1×PBS (PBS 10×, Invitrogen 70013-016). The PBS was removed and 3 ml Trypsin-EDTA (Trypsin-EDTA, Invitrogen 25200-056) was added and incubated for 15 minutes at 37° C. with a CO₂ level of 5% at a maximum humidity. After incubation, 10 ml 1×PBS with 10% FBS was added and 1 ml of the total amount was used for the cell count with the Beckman Coulter Vi-Cell.

The remaining 12 ml were centrifuged for 5 minutes at 300 G. The medium was removed and the pellet was resuspended in minimum essential medium with 20% FBS, 1% antibiotics, 1% fungizone, 2% non-essential amino acids, 2% amino acids and 2% vitamins. Approximately 4.0×10⁵ cells were added to 10, 25 cm² culture bottles and grown overnight at 37° C., 5% CO₂ and maximum humidity.

Collecting cells. The culture medium was removed and the cells were washed with 3 ml 1×PBS. 1 ml Trypsin-EDTA solution was added and cells were incubated for 15 minutes. 4 ml 1×PBS with 10% FBS was added to the trypsinized cells. The resultant cells were mixed well and 1 ml was used for a cell count and 4 ml was used for centrifugation and RNA-isolation. The supernatant was removed and the cell pellet was stored at −150° C., until ready for the RNA-isolation.

Cell count. Cells were analyzed with the Beckman Coulter Vi-Cell. Protocol: CCD 1077SK in Trypsin.

RNA-isolation. Reaction kit: QIAGEN Rneasy® mini kit (50) (QIAGEN 74104). 350 μl of buffer RLT was added with 1% β-ME to disrupt the cells. The tube was gently vortexed by hand to loosen the pellet and the cells were mixed by pipetting. The lysate was passed 5× through a 19-gauge needle (0.9 mm Ø) fitted to an Rnase-free syringe. 350 μl of 70% ethanol was added to the lysate and mixed by pipetting, and the tube was closed gently. 700 μl of sample was applied on the Rneasy mini column and placed in a 2 ml collection tube. The tube was closed gently and centrifuged for 20 seconds at 8,000 G (10,000 rpm). The flow through was discarded. 350 μl of buffer RW1 was added to the Rneasy mini column, the tube was gently closed and centrifuged for 20 s at 8,000 G (10,000 rpm). The flow through was discarded. 10 μl Dnase1 stock-solution was added to 70 μl buffer RDD, mixed well, and 80 μl was added directly onto the silica-gel membrane. The tube was gently closed and incubated at 25° C. for 30 min. 350 μl of buffer RW1 was added to the Rneasy mini column, the tube was gently closed and centrifuged for 20 seconds at 8,000 G (10,000 rpm). The flow through was discarded. After transferring the Rneasy column to a new collection tube, 500 μl buffer RPE was added onto the column and the tube was gently closed. The tube was centrifuged for 20 seconds at 8,000 G (10,000 rpm) and the flow through was discarded. Another 500 μl buffer RPE was added to the Rneasy column, the tube was gently closed and centrifuged for 2 min at 8,000 G (10,000 rpm) and the flow through was discarded. The column was dried by centrifugation for an additional minute at 8,000 G (10,000 rpm). The RNA was eluted by placing the column in a new 1.5 ml tube and pipetting 50 μl RNase-free water directly onto the membrane. The tube was gently closed and centrifuged for 1 min at 8,000 G (10,000 rpm). Step 11 was repeated with another 50 μl and the sample was eluted in the same collection tube.

Control-procedures. The optical density was measured with a Perkin Elmer MBA 2000 using the option in menu: RNA concentration at a dilution of 5 μl RNA-sample to 65 μl Rnase free water. Nuclease-free water was used as the blank sample.

Experion Automated Electrophoresis System

### RNA Standards Kit

PCR of RNA-Samples to Assess the Quality of the RNA Isolated from the Cells.

### Mastermix (7 Samples)

| | | |
|---|---|---|
| IQ SYBR Green | 87.5 μl | (BioRad 170-8880) |
| Supermix | | |
| MCP-1 forward | 8.75 μl | 5′ CTCGCCTCCAGCATGAAAGT 3′ |
| MCP-1 reverse | 8.75 μl | 5′ GTGACTGGGGCATTGATT 3′ |
| Nuclease free water | 52.5 μl | |

22.5 μl Mastermix + 2.5 μl sample/control

| | |
|---|---|
| Optimal annealing temperature: | 59° C. |

### 96-Well Plate

| | 1 | 2 | 3 |
|---|---|---|---|
| A | + | + | − |
| B | Sample | Sample | |

The plate was centrifuged after pipetting and sealing.

PCR-Protocol

| Cycle 1: (1X) | |
|---|---|
| Step 1: | 95.0° C. for 10:00 |
| Cycle 2: (35X) | |
| Step 1: | 95.0° C. for 00:30 |
| Step 2: | 59.0° C. for 00:30 |
| Step 3: | 72.0° C. for 00:30 |
| Data was collection and analyzed in real-time. | |
| Cycle 3: (1X) | |
| Step 1: | 72 .0° C. for 10:00 |
| Cycle 4: (1X) | |
| Step 1: | 50.0° C. for 01:00 |
| Cycle 5: (225X) | |
| Step 1: | 50.0° C. for 00:10 |
| Setpoint temperature was increased after cycle 2 by | |
| 0.2° C. Melt curve data was collection and analyzed in real- | |
| time. | |
| Cycle 6: (1X) | |
| Step 1: | 4.0° C. HOLD |

Allele-Specific Reverse Transcription of RNA to cDNA.

3 alternative reverse transcriptase enzymes were tested:

Thermo-X

Superscript III

Bio-Rad, MMLV

6 different primers were tested:

3 against allele A (20 nt, 15 nt, 10 nt)

3 against allele C (20 nt, 15 nt, 10 nt)

Target SNP=NCBI: rs363125
RT-Reaction with Primer for Allele A

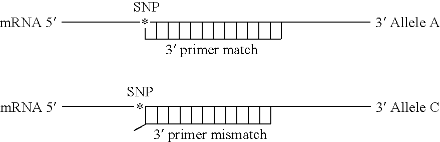

The goal was to get only the product of the allele with a perfect primer match. This was achieved by the following methods:

3′ primer end mismatch primers.
A-variant
20nt (363125A20)
SEQ ID. 52: ′ GTGTTCTTCTAGCGTTGAAT 3′
15nt (363125A15)
SEQ ID. 47: ′ CTTCTAGCGTTGAAT 3′
10nt (363125A10)
SEQ ID. 42: ′ AGCGTTGAAT 3′
C-variant
20nt (363125C20)
SEQ ID. 64: ′ GTGTTCTTCTAGCGTTGAAG 3′
15nt (363125C15)
SEQ ID. 59: ′ CTTCTAGCGTTGAAG 3′
10nt (363125C10)
SEQ ID. 54: ′ AGCGTTGAAG 3′

Reverse Transcription Protocols

Thermo-X (Reaction Set Labeled “X”)
1 Reaction mix:

| | | |
|---|---|---|
| RNA sample | 1.5 | μl |
| 2 μM sequence specific primer | 1 | μl |
| 10 mM dNTP mix | 1 | μl |
| Nuclease free H₂O | 6.5 | μl |

2 Incubated 5 minutes at 65° C. and cooled on ice for 1 minute.
3 Collected content of tube by brief centrifugation and added:

| | |
|---|---|
| 5x Thermo-X buffer | 4 μl |
| Thermo-X RT 200 U/ml | 1 μl |
| Nuclease free H₂O | 5 μl |

4 Vortexed and centrifuged briefly.
5 Incubated at 62° C. for 30 minutes.
6 Inactivated at 90° C. for 5 minutes.
7 Stored at −20° C.

Superscript III (Reaction Set Labeled “Y”)
1 Reaction mix:

| | | |
|---|---|---|
| RNA sample | 1.5 | μl |
| 2 μM sequence specific primer | 1 | μl |
| 10 mM dNTP mix | 1 | μl |
| Nuclease free H₂O | 9.5 | μl |

2 Incubated 5 minutes at 65° C. and cooled on ice for 1 minute.
3 Collected content of tube by brief centrifugation and added:

| | |
|---|---|
| 5x first strand buffer | 4 μl |
| Superscript III RT 200 U/ml | 2 μl |
| 0.1 M DTT | 1 μl |

4 Mixed by pipetting
5 Incubated at 55° C. for 60 minutes.
6 Inactivated at 70° C. for 15 minutes.
7 Stored at −20° C.

Bio-Rad iScript cDNA Synthesis Kit, Reverse Transcriptase (MMLV Derived); Reaction Set Labeled “Z”.
1 Reaction mix:

| | | |
|---|---|---|
| RNA sample | 1.5 | μl |
| 2 μM sequence specific primer | 1 | μl |
| 10 mM dNTP mix | 1 | μl |
| iScript reverse transcriptase | 1 | μl |
| Nuclease free H₂O | 15.5 | μl |

2 Incubated 5 minutes at 25° C.
3 Incubated 30 minutes at 42° C.
4 Incubated 5 minutes at 85° C.
5 Stored at −20° C.

Bio-Rad iScript cDNA Synthesis Kit, Via Bio-Rad Protocol.
1 Reaction mix:

| | | |
|---|---|---|
| RNA sample | 1.5 | μl |
| 5x iScript reaction mix | 4 | μl |
| iScript reverse transcriptase | 1 | μl |
| Nuclease free H₂O | 13.5 | μl |

2 Incubated 5 minutes at 25° C.
3 Incubated 30 minutes at 42° C.
4 Incubated 5 minutes at 85° C.
5 Stored at −20° C.

PCR of the Reverse Transcribed cDNA Samples Mastermix (64 Samples)

| | | |
|---|---|---|
| Accuprime | 32 | μl |
| Upper primer 8 μM 173U19 | 38.4 | μl |
| Lower primer 8 μM 436L20 | 38.4 | μl |
| Buffer A | 320 | μl |
| SYBR Green I (dil. 40000x) | 640 | μl |
| H₂O | 403.2 | μl |

173U19
′ GCCTTCGAGTCCCTCAAGT 3′
436L20
′ GACAATGATTCACACGGTCT 3′

23 μl mastermix and 2 μl sample per well.

Plate Setup

| | 1 | 2 | 3 | 4 | 5 | 6 | 7 | 8 | 9 | 10 | 11 | 12 |
|---|---|---|---|---|---|---|---|---|---|---|---|---|
| A | XA10 | XA15 | XA20 | | YA10 | YA15 | YA20 | | ZA10 | ZA15 | ZA20 | |
| B | XA10 | XA15 | XA20 | | YA10 | YA15 | YA20 | | ZA10 | ZA15 | ZA20 | |
| C | XA10 | XA15 | XA20 | | YA10 | YA15 | YA20 | | ZA10 | ZA15 | ZA20 | |
| D | XC10 | XC15 | XC20 | | YC10 | YC15 | YC20 | | ZC10 | ZC15 | ZC20 | |
| E | XC10 | XC15 | XC20 | | YC10 | YC15 | YC20 | | ZC10 | ZC15 | ZC20 | |
| F | XC10 | XC15 | XC20 | | YC10 | YC15 | YC20 | | ZC10 | ZC15 | ZC20 | |
| G | | | | | | | | | | | | |
| H | Control | Control | Control | | | | | | | | | |

Example of the labeling used above to indicate what reaction product was placed in what well of the plate: XA10 means the reaction product from reaction set “X”, using the primer specific for the “A” allele that was of length 10 nucleotides (that is, reaction using Thermo-X enzyme, and the primer corresponding to SEQ NO. 42 as described.

PCR protocol

| Cycle 1: (1X) | |
|---|---|
| Step 1: | 95.0° C. for 03:00 |
| Cycle 2: (35X) | |
| Step 1: | 95.0° C. for 00:30 |
| Step 2: | 57.0° C. for 00:30 |
| Step 3: | 72.0° C. for 00:30 |
| Data was collected and analyzed. | |
| Cycle 3: (1X) | |
| Step 1: | 72.0° C. for 10:00 |
| Cycle 4: (1X) | |
| Step 1: | 50.0° C. for 01:00 |
| Cycle 5: (225X) | |
| Step 1: | 50.0° C. for 00:10 |
| Setpoint temperature was increased after cycle 2 by | |
| 0.2° C. Melt curve data was collected and analyzed. | |
| Cycle 6: (1X) | |
| Step 1: | 4.0° C. HOLD |

### Results

The RNA-isolation resulted in pure RNA according to the Experion, OD measurement and the control PCR. The PCR of the reverse transcription samples showed that Superscript III was more effective than Bio-Rad iScript cDNA synthesis kit, reverse transcriptase and Thermo-X reverse transcriptase. The amplification curves of the Superscript III samples did not show as much difference in threshold cycles between the triple measurements as the other 2 reverse transcriptases.

The Bio-RAD iScript cDNA synthesis kit, reverse transcriptase showed no amplification signal at all. This could be caused by the absence of the iScript reaction mix which contains several elements that could be involved with the amplification of a sample.

Electrophoresis of the PCR-results showed the following: (1) The positive control showed 2 bands one at 280 bp and one at 370 bp this means that both alleles were present. (2) The electrophoresis of the PCR-results showed that reactions YA15 and YC15 gave the best results because there was only one band visible in each sample (each a different allele). (3) The set of reactions using Thermo-X (called the “X” reaction set) also showed some specificity. (4) The size of the PCR product from the PCR reaction using the cDNA YA15 was about 280 bp and the size of YC15 was about 370 bp, from this can be concluded that the allele with a “C” in SNP NCBI: rs363125 has a longer PCR product in this patient from the region of the CAG repeat.

### Conclusion

These data clearly indicate successful allele-specific reverse transcription reaction by amplifying only one of two alleles of the HD gene. It can therefore be concluded that in this patient the allele containing the disease-causing expansion of the CAG repeat region is the allele with a C at SNP site NCBI: rs363125, and not the allele with an A at SNP site NCBI: rs363125. It can further be concluded that an allele-specific therapy using an siRNA targeting the allele with a C at SNP site NCBI: rs262125 would be an appropriate therapy to administer to this patient to prevent or treat the patient's disease.

### Example 2

The following hypothetical example teaches how to use the method and materials of the subject invention to identify the disease-causing allele and apply an allele-specific therapy to a patient in the case in which the SNP site use for the allele-specific therapy lies 5′ to the disease-causing mutation in the relevant gene. Suppose that there is a gene for which the mRNA contains the following elements, in 5′ to 3′ sequence:
1. A SNP site “X” for which the allele variants have a C or a T at the site, and an allele-specific therapy is available such that either the C allele or the T allele can be targeted by a therapy to suppress the translation of the mRNA into protein (for example, by resulting in the selective destruction of the mRNA of one allele).
2. A disease-causing mutation that is downstream (in the 3′ direction) of the aforementioned SNP site “X”.
3. A SNP site “Y” that is downstream (in the 3′ direction) of the disease-causing mutation for which (for example) the allele variants have a G or a T at the site.

Given this situation, the method of the subject invention may be used to identify which allele contains the disease-causing mutation and which allele-specific therapy to administer to the patient to treat the disease, as follows:
1. An allele-specific reverse transcription primer is used to selectively reverse transcribe the mRNA of the allele variant that has a “G” at SNP site “Y”.
2. The resulting cDNA product of the reverse transcription is used as a template in a PCR reaction that amplifies the region potentially containing the disease-causing mutation, and the PCR product is analyzed to determine the presence or absence of the disease-causing mutation in the PCR reaction product. Suppose, for this example, that the analysis shows that the disease-causing mutation is present on this allele.
3. In addition, the resulting cDNA product of the reverse transcription is used as a template in a PCR reaction that amplifies the region containing SNP site “X” and the PCR product is sequenced to determine whether the nucleotide base at the SNP site “X” is a “C” or a “T” on this allele. Suppose, for this example, that the analysis shows that the SNP site “X” has a “T” on this allele. Given these results, the patient's gene contains a T at SNP site “X”, the disease-causing mutation, and a “G” at SNP site “Y” all on the same allele.
4. In addition, an allele-specific reverse transcription primer is used to selectively reverse transcribe the mRNA of the allele variant that has a “T” at SNP site “Y”.
5. The resulting cDNA product of this second reverse transcription reaction is used as a template in a PCR reaction that amplifies the region potentially containing the disease-causing mutation, and the PCR product is analyzed to determine the presence or absence of the disease-causing mutation in the PCR reaction product. In this example, in a heterozygous patient, the analysis should show that the disease-causing mutation is not present on this allele.
6. Furthermore, the resulting cDNA product of the second reverse transcription reaction is used as a template in a PCR reaction that amplifies the region containing SNP site “X” and the PCR product is sequenced to determine whether the nucleotide base at the SNP site “X” is a “C” or a “T” on this allele. Suppose, for this example, that the analysis shows that the SNP site “X” has a “C” on this allele. Therefore, one allele of the gene in this patient contains a C at SNP site “X”, no disease-causing mutation, and a “T” at SNP site “Y,” all on the same allele.
7. From these results, it would be concluded that the allele-specific treatment that targets the “T” allele at SNP site “X” should be used to treat this patient's disease.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

What is claimed:

1. A method for determining which single nucleotide polymorphism variant of an allele from a gene isolated from a heterozygous patient is on the same mRNA transcript as a disease-causing mutation at a remote region of the gene's mRNA comprising:
a) an allele-specific reverse transcription reaction using an allele-specific primer which recognizes one single nucleotide polymorphism variant, and
b) analysis of an allele-specific cDNA product from the allele-specific reverse transcription reaction at the remote region of the gene to determine the presence or absence of the mutation on the allele-specific cDNA product.

2. The method of claim 1, wherein the 3′ end of the allele-specific primer is positioned at the single nucleotide polymorphism nucleotide position.

3. A method of claim 1 further comprising:
c) at least a second allele-specific reverse transcription reaction using at least a second allele-specific primer which recognizes at least a second single nucleotide polymorphism variant, and
d) analysis of at least a second allele-specific cDNA product from at least the second allele-specific reverse transcription reaction at the remote region of the gene to determine the presence or absence of the mutation on at least the second allele-specific cDNA product.

4. A method of claim 2 further comprising:
c) at least a second allele-specific reverse transcription reaction using at least a second allele-specific primer which recognizes at least a second single nucleotide polymorphism variant, and
d) analysis of at least a second allele-specific cDNA product from at least the second allele-specific reverse transcription reaction at the remote region of the gene to determine the presence or absence of the mutation on at least the second allele-specific cDNA product.

5. The method of claim 3 wherein the 3′ end of at least the second allele-specific primer is positioned at the single nucleotide polymorphism nucleotide position.

6. The method of claim 4 wherein the 3′ end of at least the second allele-specific primer is positioned at the single nucleotide polymorphism nucleotide position.

7. A method of treating a patient comprising:
a) determining which single nucleotide polymorphism variant is on the same mRNA transcript as a disease causing mutation according to the method of claim 1, and
b) applying an allele-specific therapy to the single nucleotide polymorphism variant.

8. The method of claim 7, wherein the allele-specific therapy comprises allele-specific RNA interference using siRNA or shRNA.

9. The method of claim 1, wherein the patient is at risk of developing a neurodegenerative disorder selected from the group consisting of Dystophia myotonica, Spinocerebellar ataxia type 1, Spinocerebellar ataxia type 2, Spinocerebellar ataxia type 3, Spinocerebellar ataxia type 6, Spinocerebellar ataxia type 7, Spinocerebellar ataxia type 8, Spinocerebellar ataxia type 17, Huntington disease-like 2, Spinal and bulbar muscular atrophy, Huntington's disease, Dentatorubral-pallidoluysian atrophy, Oculopharyngeal dystrophy, Congenital central hypoventilation syndrome, Infantile spasms, Synpolydactyl), Cleidocranial dysplasia, Holoprosencephaly, Hand-foot-genital syndrome, Type II blephorophimosis, ptosis, and epicanthus inversus syndrome.

10. The method of claim 7, wherein the allele-specific therapy operates at the same single nucleotide polymorphism site used to determine which mRNA transcript contains the disease-causing allele in said patient.

11. The method of claim 7, wherein the allele-specific therapy operates at a different single nucleotide polymorphism site than the site used to determine which mRNA transcript contains the disease-causing allele in said patient.

12. A kit for determining which single nucleotide polymorphism variant of an allele of a heterozygous patient is on the same mRNA transcript as a disease-causing mutation located at a remote region of the gene's mRNA comprising
a) an allele-specific primer which recognizes one single nucleotide polymorphism variant, and
b) a set of instructions.

13. The kit of claim 12, further comprising: at least a second allele-specific primer which recognizes at least a second single nucleotide polymorphism variant.

14. The kit of claim 12, wherein the 3′ end of the allele-specific primer is positioned at the single nucleotide polymorphism nucleotide position.

15. The kit of claim 12, wherein the 3′ end of at least the second allele-specific primer is positioned at the single nucleotide polymorphism nucleotide position.

16. The kit of claim 13, wherein the 3′ end of at least the second allele-specific primer is positioned at the single nucleotide polymorphism nucleotide position.

17. The kit of claim 14, wherein the 3′ end of at least the second allele-specific primer is positioned at the single nucleotide polymorphism nucleotide position.

18. The kit of claim 12, wherein the allele-specific primer is selected from the group consisting of SEQ. ID. 1-SEQ. ID. 84.

19. The kit of claim 13, wherein
at least the second allele-specific primer is selected from the group consisting of SEQ. ID. 11-SEQ. ID. 20 if the allele-specific primer is selected from the group consisting of SEQ. ID. 1-SEQ. ID. 10; or
at least the second allele-specific primer is selected from the group consisting of SEQ. ID. 31-SEQ. ID. 40 if the allele-specific primer is selected from the group consisting of SEQ. ID. 21-SEQ. ID. 30; or
at least the second allele-specific primer is selected from the group consisting of SEQ. ID. 53-SEQ. ID. 64 if the allele-specific primer is selected from the group consisting of SEQ. ID. 41-SEQ. ID. 52; or
at least the second allele-specific primer is selected from the group consisting of SEQ. ID. 75-SEQ. ID. 84 if the allele-specific primer is selected from the group consisting of SEQ. ID. 65-SEQ. ID. 74.

20. The kit of claim 18, wherein the allele-specific primer is selected from the group consisting of SEQ. ID. 42, SEQ. ID. 47, SEQ. ID. 52, SEQ. ID. 54, SEQ. ID. 59, and SEQ. ID. 64.
